(19) United States
(12) Patent Application Publication
(10) Pub. No.: US 2003/0074862 A1
(43) Pub. Date: Apr. 24, 2003

(54) Method of steam sterilisation of medical products

(76) Inventor: Heinrich Lohmuller, Rangendingen (DE)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/169,460

(22) PCT Filed: Mar. 12, 2001
(86) PCT No.: PCT/IB01/00409

(30) Foreign Application Priority Data
Apr. 12, 2000 (SE) 0001343-3

(51) Int. Cl. B65B 55/02

(52) U.S. Cl. 53/425

(57) ABSTRACT

A method of steam sterilisation of medical products is described, in which the medical products are placed separately or in multiples in a cardboard package, the cardboard package is sealed, and the medical products are subsequently steam sterilised in the sealed cardboard package.

The present invention concerns a method of steam sterilisation of medical products, in particular a method of steam sterilisation of medical products in an autoclave.

### TECHNICAL BACKGROUND

Medical products are currently sterilised using differing methods. One method for example is steam sterilisation of the medical products. This is an effective and recognised sterilisation method which is primarily used for the sterilisation of for example disposable medical products. This is due in particular to its better environmental compatibility, for example in contrast to sterilisation methods using gamma radiation or ETO.

With steam sterilisation of medical products, these are set up separately in stainless steel racks, and the stainless steel racks with the medical products are subsequently introduced into an autoclave for example. There, the medical products are heat sterilised with steam in the autoclave chamber in a known manner. For this, the medical products are typically arranged in a flexible and at least partly steam-permeable and bacteria-proof package.

Following the sterilisation cycle in the autoclave, the racks with the medical products are removed from the autoclave chamber. After a cooling period which may be required in certain circumstances, the medical products are manually unloaded from the rack and put into a cardboard package. An information leaflet and/or operating instructions are enclosed with the medical products and the cardboard package is sealed. Lastly a label is also stuck on the cardboard package so that this is ready for delivery and sale.

Although steam sterilisation is currently the preferred method with medical products and although it has essentially no environmentally damaging aspects, it does however have certain disadvantages. Firstly it is time consuming and labour intensive, since the medical products have to be manually put into, or set up in, the sterilisation racks, and after sterilisation in the autoclave have again to be manually taken out of the sterilisation rack and put into a cardboard package. Secondly there is an increased germ-loading, even when the medical products are each separately packed, since they have to be manually handled after sterilisation and are exposed to the surroundings for a long time before being put into the cardboard package.

If the medical products are each packed separately, bacteria can reach the outside of the package during the manual handling and can become attached and under certain circumstances can multiply. When the package is then later opened in order to use the medical products, the bacteria can become detached from the outside and reach the medical product which thereby becomes contaminated. If the medical products are not each separately packed, they can be directly contaminated during manual handling.

Furthermore there is high energy consumption because, apart from the unavoidable creation of the steam, the sterilisation racks have to be heated up to the sterilisation temperature each time.

### DESCRIPTION OF THE INVENTION

Against this background the object of the present invention is to provide a method of steam sterilisation of medical products, which is time-saving, labour-saving and energy-saving and consequently allows a low cost steam sterilisation of medical products.

This object is solved by a method of steam sterilisation of medical products, in which the medical products are put into a cardboard package separately or in multiples, the cardboard package is sealed, and the medical products are then steam sterilised in the sealed cardboard package.

With this new method, a safe, and at the same time low cost, steam sterilisation of medical products is possible. The medical products are directly placed into the cardboard package, an information leaflet and/or operating instructions are enclosed, the cardboard package is sealed and a label is stuck on the package. Then the sealed cardboard package with the medical products contained therein is sterilised with steam.

In this way firstly the manual handling of the medical products during setting up in the sterilisation rack and also during removal from the sterilisation rack is avoided. Secondly, only one sterilisation rack is needed for the cardboard packages, since the medical products are arranged or set up in the cardboard packages and the entire sealed packages with the medical products contained therein are steam sterilised. This rack for the cardboard packages can however be significantly lighter, i.e. of a lighter construction and made of less material, since it only has to accommodate a few cardboard packages and not a large number of medical products. In this way however, the time required to heat up the sterilisation rack is reduced and consequently less energy is required.

Altogether a method of steam sterilisation of medical products is thereby provided which requires less preparation time and less time afterwards, since the medical products do not have to be sorted into a sterilisation rack or taken out of this any more. In this way the need for personnel is markedly reduced. Additionally, the risk is removed that the sterilised medical products are carelessly handled or even dropped and thereby damaged or contaminated. Also the processing time itself is reduced, since the sterilisation rack is more rapidly heated up to the sterilisation temperature. This causes however at the same time a significant energy-saving in the whole sterilisation process. Finally, the germ-loading on the medical products is significantly reduced, since these do not have to be manually handled after sterilisation any more but instead are directly present in the cardboard package, and thus no longer exposed to the surrounding atmosphere.

A further advantage of the new method is that it can be used for any type of medical products. For example, medical tubes and conduits, connectors, cannulas, syringes, filters for dialysis (dialysers) with hollow fibre membranes or flat membranes and also medical fluids and powder concentrates can be steam sterilised, as well as in principle such medical products whose material properties allow heating up to the sterilisation temperature. It should be observed at this juncture that the above listing of medical products is not to be understood as limiting and that the method can also be used for other products and objects.

The method is however especially advantageous with disposable medical products and is thus used for disposable medical products in accordance with a preferred embodiment, which products are in a flexible and at least partly steam-permeable and bacteria-proof package.

To ensure the sterility of the sterilised medical products in the cardboard package also for long periods of time it is also possible, and provided in accordance with a further embodiment of the invention, that the medical products are placed in a cardboard package of bacteria-proof material.

This is especially useful when the disposable or reusable medical products are not each in a flexible and at least partly steam-permeable and bacteria-proof package.

Advantageously, the steam sterilisation of the medical products in the cardboard package is carried out, in accordance with a preferred embodiment, in an autoclave. In this way, the method is further simplified, since autoclaves are widely available and steam sterilisation of objects in autoclaves and the operation of autoclaves is basically known to the skilled person. This person will consequently have no difficulties in utilising the method of steam sterilisation of medical products, which is explained more thoroughly below.

The new method of steam sterilisation of medical products contained in a sealed cardboard package comprises the following method steps:

Pre-heating the medical products at an over-pressure in the autoclave chamber (a),

Producing a fractionated pre-vacuum in the autoclave chamber (b),

Heating the medical products (c),

Sterilisation of the medical products at an over-pressure in the autoclave chamber (d),

Producing a fractionated post-vacuum in the autoclave chamber (e),

Equalising the pressure of the autoclave chamber with the surrounding atmosphere (f),

In order to pre-heat the medical products, the air in the autoclave chamber is preferably heated up to a temperature of more than 100° C., more preferably to a temperature of about 110° C. In this way, the condensation of the air humidity on the medical products to be sterilised as well as on the cardboard package is prevented. This is important so that the cardboard package does not become damp and as a result of the humidity lose its shape or durability or even be destroyed.

In this connection it has been found advantageous to pre-heat the medical products at an over-pressure in the autoclave chamber, whereby the over-pressure is preferably about 2 bar.

The pre-heating of the air in the autoclave chamber preferably occurs by means of a heat exchanger, more preferably by means of an internal heat exchanger.

In order to ventilate the medical products and the cardboard package without problem and to extract all inert gases out of the autoclave chamber, a fractionated pre-vacuum is produced in the autoclave chamber, i.e. several vacuum cycles are run.

It has been found advantageous to produce a fractionated pre-vacuum of less than 400 mbar in the autoclave chamber, especially advantageous are five cycles of about 200 mbar, whereby between the vacuum cycles a pressure of more than 1 bar is produced in the autoclave chamber by means of steam impulses. For this, a pressure of preferably about 1100 mbar is produced in the autoclave chamber.

For the actual sterilisation of the medical products, these are preferably heated by means of pure steam to more than 110° C., whereby heating to about 121° C. has been found to be especially effective. To achieve sufficient sterilisation, the medical products are hereby held at about 121° C. for at least 15 minutes. More certain results are however achieved when the medical products are held at about 121° C. for about 20 minutes.

It should be noted at this juncture that dependent on the products to be sterilised, if these for example withstand higher temperatures, higher temperatures can also be chosen and thereby shorter sterilisation times.

In order to ensure that the cardboard package is completely dry, a fractionated post-vacuum is also produced to cool the medical products, whereby preferably a post-vacuum of less than 400 mbar is produced, and particularly advantageous is when a post-vacuum of about 280 mbar in two cycles is produced. It has been found advantageous to apply an air impulse of more than 700 mbar and a temperature of between 15° C. and 30° C. between the vacuum cycles in the autoclave chamber, whereby an air impulse of about 800 mbar and a temperature of about 23° C. is especially advantageous.

For pressure equalisation of the autoclave chamber with the surrounding atmosphere, this is finally brought to the surrounding atmospheric pressure.

### DESCRIPTION OF THE DRAWING

The method will now be explained in more detail using a preferred embodiment and with reference to the accompanying drawing. This shows:

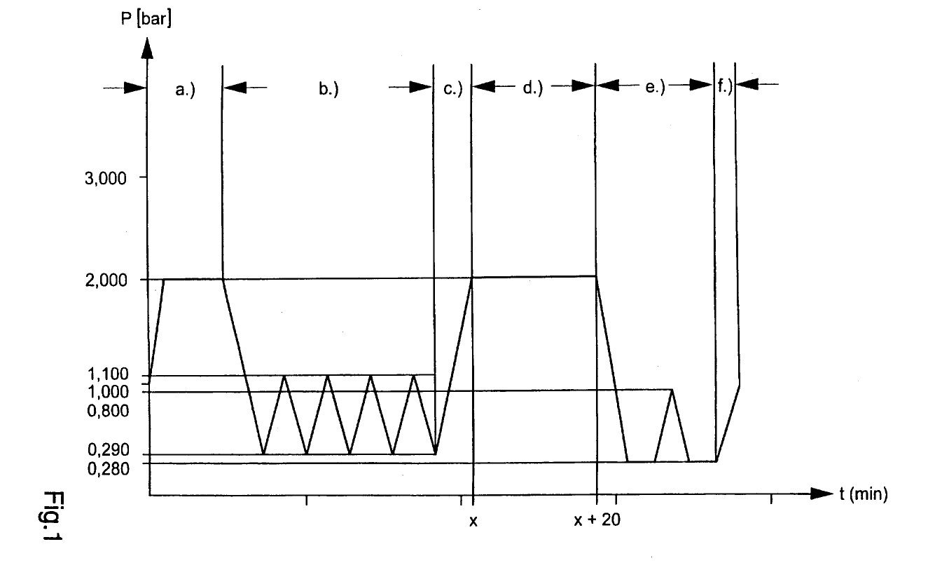

in FIG. 1 a schematic representation of the pressure change against time, and

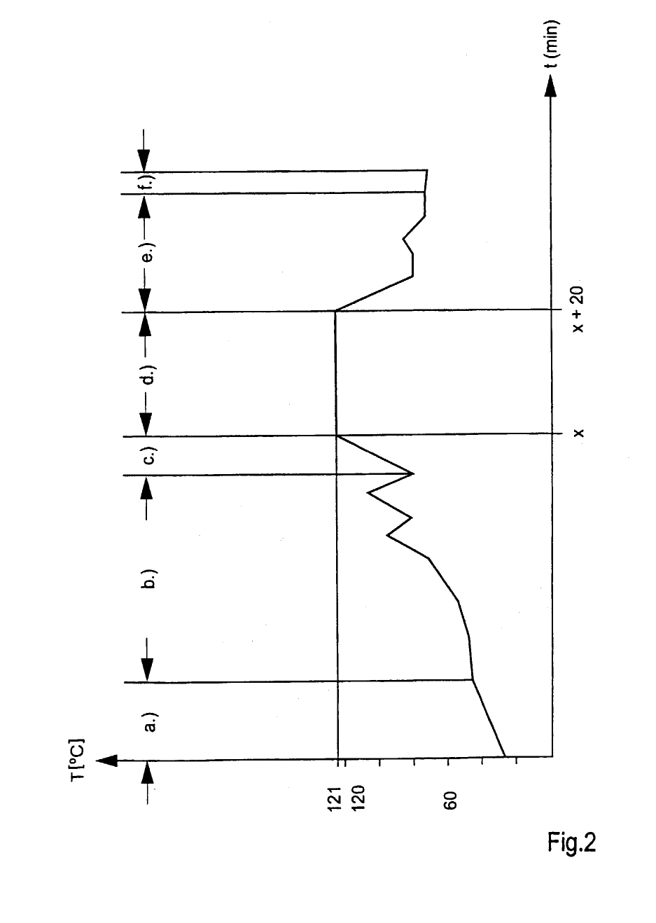

in FIG. 2 a schematic representation of the temperature change against time.

### DESCRIPTION OF A PREFERRED EMBODIMENT

FIG. 1 schematically represents the pressure change against time in the autoclave chamber with steam sterilisation of dialysers having hollow fibre membranes as disposable medical products according to the method described herein, in which the dialysers are separately contained in a flexible and partly steam-permeable and bacteria-proof package.

The dialysers are pre-heated in the first phase (a) at an over-pressure of about 2 bar. The pre-heating of the dialysers is done using the air in the autoclave chamber, which air is heated by an internal heat exchanger to about 110° C. As explained in detail above, by heating the air in the autoclave chamber the condensation of the air humidity on the dialysers as well as on the cardboard package is avoided.

In the second phase (b) a fractionated pre-vacuum is produced in the autoclave chamber, i.e. several vacuum cycles are run. As is easily identifiable from the schematic representation in FIG. 1, five cycles are run, whereby respectively a pre-vacuum of about 290 mbar is produced in the autoclave chamber. Between the vacuum cycles, steam impulses are introduced into the autoclave chamber, whereby the pressure in the autoclave chamber respectively rises again to about 1.1 bar.

After the last pre-vacuum cycle, the dialysers in the autoclave chamber are heated during phase (c) up to the sterilisation temperature. To this end, pure steam is introduced into the autoclave chamber so that the pressure rises to about 2 bar and the dialysers are heated to about 121° C. The pressure in the autoclave chamber is then held at approximately 2 bar for about 20 minutes (phase (d)), while at the same time the dialysers are held at about 121° C. This is the actual sterilisation phase.

After sterilisation of the dialysers in phase (d), a fractionated post-vacuum is produced in the autoclave chamber in phase (e) so as to cool the dialysers. As can be easily understood from the schematic representation in FIG. 1 a post-vacuum of about 280 mbar in two cycles is produced, whereby the pressure in the autoclave chamber is brought to about 800 mbar between the two cycles by introducing an air impulse into the autoclave chamber. The air impulse hereby introduced into the autoclave chamber has a temperature of about 23° C. which corresponds substantially to the surrounding temperature. In this way it is ensured that the cardboard package is completely dry after the sterilisation process.

Finally the pressure in the autoclave chamber is brought in phase (f) to the atmospheric surrounding pressure.

In FIG. 2, the temperature change against time for the method described herein for the steam sterilisation of dialysers is schematically represented. As already mentioned, the dialysers are normal dialysers with hollow fibre membranes and the shown temperature change corresponds to the temperature change in the hollow fibre bundle.

As is easily understood in this schematic representation, the dialysers are pre-heated slowly during the first phase (a). As mentioned, the pre-heating of the dialysers occurs by means of the air present in the autoclave chamber, which air is heated to about 110° C. by the internal heat exchanger. In this way, the condensation of the air humidity onto the dialysers and onto the cardboard package is prevented.

In the second phase (b) in which, as explained, a fractionated pre-vacuum is produced in the autoclave chamber, the dialysers are heated further. This heating occurs by means of steam impulses introduced into the autoclave chamber between the vacuum cycles. The inconstant temperature change in this phase (b) is as a result of this.

In the third phase (c), the dialysers are heated to the sterilisation temperature of about

121° C. The heating occurs by means of pure steam which is introduced into the autoclave chamber. As explained with reference to FIG. 1, the pressure hereby rises in the autoclave chamber to about 2 bar. The dialysers are then held at approximately 121° C. for about 20 minutes, phase (d). This is the actual sterilisation phase.

After sterilisation of the dialysers in phase (d), the dialysers are cooled in phase (e). In phase (f), in which the pressure in the autoclave chamber is brought to surrounding atmospheric pressure, the temperature drops further.

In the embodiment described herein, the dialysers are heat sterilised with steam in an autoclave which is equipped with fans in the autoclave chamber. These fans were operating respectively in phases (a), (b), (c) and (d) and assisted the uniform heating of the medical products by circulation of the air and the steam in the autoclave chamber.

1. Method of steam sterilisation of medical products, in which the medical products are placed separately or in multiples in a cardboard package, the cardboard package is sealed, and the medical products are subsequently steam sterilised in the sealed cardboard package.

2. Method according to claim 1, characterized in that the medical products are disposable medical products, which are present respectively in a flexible and at least partly steam-permeable and bacteria-proof package.

3. Method according to claim 1 or 2, characterized in that the medical products are placed in a cardboard package of bacteria-proof material.

4. Method according to any one of claims 1, 2 or 3 characterized in that the steam sterilisation of the medical products in the sealed cardboard package occurs in an autoclave.

5. Method according to claim 4, characterized in that the heat sterilisation of the medical products in the sealed cardboard package comprises the following steps:
Pre-heating the medical products at an over-pressure in the autoclave chamber (a.),
Producing a fractionated pre-vacuum in the autoclave chamber (b.),
Heating the medical products (c.),
Sterilisation of the medical products at an over-pressure in the autoclave chamber (d.),
Producing a fractionated post-vacuum in the autoclave chamber (e.),
Equalising the pressure of the autoclave chamber with the surrounding atmosphere, (f.).

6. Method according to claim 5, characterized in that for pre-heating the medical products, the air in the autoclave chamber is heated to a temperature of more than 100° C.

7. Method according to claim 6, characterized in that for pre-heating the medical products, the air in the autoclave chamber is heated to a temperature of about 110° C.

8. Method according to one of claims 6 or 7, characterized in that the medical products are pre-heated at an over-pressure in the autoclave chamber.

9. Method according to claim 8, characterized in that the medical products are pre-heated at an over-pressure of about 2 bar in the autoclave chamber.

10. Method according to any one of claims 6 to 9, characterized in that the air in the autoclave chamber is heated by means of a heat exchanger.

11. Method according to claim 10, characterized in that the air in the autoclave chamber is heated by means of an internal heat exchanger.

12. Method according to claim 5, characterized in that a fractionated pre-vacuum of less than 400 mbar is produced in the autoclave chamber.

13. Method according to claim 12, characterized in that that a fractionated pre-vacuum of about 290 mbar in 5 cycles is produced in the autoclave chamber

14. Method according to one of claims 12 or 13, characterized in that between the vacuum cycles steam impulses are introduced into the autoclave chamber, so that a pressure of more than 1 bar is produced in the autoclave chamber.

15. Method according to claim 14, characterized in that between the vacuum cycles steam impulses are introduced into the autoclave chamber, so that a pressure of about 1100 mbar is produced in the autoclave chamber.

16. Method according to claim 5, characterized in that the medical products are heated to more than 110° C. by means of pure steam.

17. Method according to claim 16, characterized in that the disposable products are heated to about 121° C. by means of pure steam.

18. Method according to one of claims 16 or 17, characterized in that the medical products are held for longer than 15 minutes at about 121° C.

19. Method according to claim 18, characterized in that the medical products are held at approximately 121° C. for about 20 minutes.

20. Method according to claim 5, characterized in that a fractionated post-vacuum of less than 400 mbar is produced.

21. Method according to claim 20, characterized in that a fractionated post-vacuum of about 280 mbar in two cycles is produced.

22. Method according to one of claims 20 or 21, characterized in that by means of an air impulse which is introduced into the autoclave chamber between vacuum cycles, a pressure of more than 700 mbar is produced in the autoclave chamber.

23. Method according to claim 22, characterized in that by means of an air impulse which is introduced into the autoclave chamber between vacuum cycles, a pressure of about 800 mbar is produced in the autoclave chamber.

24. Method according to claim 22 or 23, characterized in that the air introduced into the autoclave chamber has a temperature of between 15° C. and 30° C.

25. Method according to claim 24, characterized in that the air introduced into the autoclave chamber has a temperature of about 23° C.

26. Method according to claim 5, characterized in that the autoclave chamber is brought to atmospheric pressure.
